(19) United States
(12) Patent Application Publication
(10) Pub. No.: US 2026/0209819 A1
(43) Pub. Date: Jul. 23, 2026

(54) METHOD FOR DETERMINING A GUT MICROBIOME ALTERATION

(71) Applicant: Compagnie Gervais Danone, Glf-sur-Yvette (FR)

(72) Inventors: Muriel DERRIEN, Bures sur Yvette (FR); Julien TAP, Massy (FR); Patrick VEIGA, Puteaux (FR)

(21) Appl. No.: 19/137,202

(22) PCT Filed: Dec. 9, 2022
(86) PCT No.: PCT/IB2022/000720

§ 371 Date: Jun. 9, 2025

(51) Int. Cl. C12Q 1/06; A23L 33/135; A23L 33/15; A23L 33/21

(52) U.S. Cl. CPC C12Q 1/06; A23L 33/135; A23L 33/15; A23L 33/21; G01N 2800/06

(57) ABSTRACT

The present invention relates to a method for determining whether an individual has or is at risk of having an alteration of the gut bacterial diversity.

### FIELD OF THE INVENTION

The present invention relates to a method for determining whether an individual has or is at risk of having an alteration of the gut bacterial diversity.

### TECHNICAL BACKGROUND

The microbes inhabiting the human gastrointestinal tract have been estimated to equal or exceed the host's cells. Some studies even suggest that they could outnumber the host's cells by 10 times and the host's genes by more than 100 times. These digestive-tract associated microbes are commonly referred to as the gut microbiota, a complex and dynamic ecosystem comprising thousands of bacterial species, among which commensal, beneficial or pathogen bacteria.

Due to its specific biochemical interaction with the host and its systemic integration into the host biology, the gut microbiota is often compared to an organ. Like any other organ, the human gut microbiota plays a crucial role in both health and diseases. While a normal state of the microbiota seems to guaranty homeostasis, an imbalance or a loss of part of this microbiota correlate with many diseases, including obesity, inflammatory bowel disease (IBD), irritable bowel syndrome (IBS), type 2 diabetes, non-alcoholic liver disease, cardio-metabolic diseases, allergies and malnutrition.

Many factors, either exogenous or endogenous, affect the composition of the gut microbiota. These factors include diet, host genotype, antibiotics history, age and sex. Nevertheless, recent studies have shown that environmental and host factors explain less than 20% of the variation in microbial composition, suggesting significant roles for stochastic factors and ecological rules in gut microbiome assembly. A factor known to influence species distribution and diversity of ecosystems is the history of community assembly. Indeed, members' appearance order in the ecosystem influences the evolution of the ecosystem, a phenomenon known as the priority effect, which has been studied in animal models but remains underexplored in human due to the lack of datasets that are both large-scale and longitudinally dense. Altogether, multiple factors contribute to the large inter-individual variability of human gut microbiota across the life span. This large variation among subjects, combined with individualized stability, justifies understanding better underlying ecological features to guide microbiome-based preventive or therapeutic approaches.

However, assessing and understanding the gut bacterial diversity remain extremely difficult due to the lack of reliable scientific tools or comprehensive methods applicable to most people.

### SUMMARY OF THE INVENTION

The present invention follows from the unexpected finding made by the Inventors that the ratio of Faecalibacterium to Bacteroides correlates with the diversity of the gut microbiota and could be used as a relevant biomarker to detect or predict an alteration of the gut microbiota.

Accordingly, the present invention provides a method for determining whether an individual has or is at risk of having an alteration of the gut bacterial diversity by determining the ratio of Faecalibacterium population/Bacteroides population in a microbiota sample from said individual.

Such a determination is useful, in particular for assessing whether an individual is at risk of developing a pathology, such as obesity, inflammatory bowel disease (IBD), irritable bowel syndrome (IBS), type 2 diabetes, non-alcoholic liver disease, cardio-metabolic diseases, allergies or malnutrition.

### DETAILED DESCRIPTION OF THE INVENTION

As used herein, the term “individual” shall be taken to mean a mammal, preferably any human individual. In the context of the invention, an individual can be healthy or unhealthy. The individual can be an adult, an elderly person, a child or a newborn.

As used herein, the expression “alteration of the gut bacterial diversity” designates a disruption between the types of organisms present in the gut microbiota of an individual. Such an alteration can lead to a gut dysbiosis, which is defined as an imbalance of the composition of the microbiota, which is linked to the pathogenesis of both intestinal and extra-intestinal disorders.

By “microbiota”, it is herein referred to the microflora present in intestines. In microbiology, flora refers to the collective bacteria and other microorganisms in an ecosystem (e.g., some part of the body of an animal host). The “gut microbiota” consists of all the species present in the gut of an individual.

As used herein the term “relative abundance” refers to the relative quantification of a plurality of taxonomic profiles of the microbiota e.g. by means of ratio, or %, such as Taxon A 50% Taxon B 50% (taxa equally abundant); Taxon A 40% Taxon B 30% Taxon C 30% (taxon A the most abundant population).

As used herein, the term “Faecalibacterium” refers to the bacterial genus Faecalibacterium. Its sole known species is Faecalibacterium prausnitzii.

As used herein, the term “Bacteroides” refers to the bacterial genus Bacteroides. Its type species are Bacteroides fragilis, Bacteroides vulgatus and Bacteroides stercoris, in particular B. fragilis.

The “ratio of Faecalibacterium population/Bacteroides population” refers to the number of bacteria in the microbiota (or in the microbiota sample) belonging to the genus Faecalibacterium divided by the number of bacteria in the microbiota (or in the microbiota sample) belonging to the genus Bacteroides. The number of bacteria belonging to the Faecalibacterium or Bacteroides genera can be estimated or quantified by techniques that are well known by one skilled in the art. Such techniques include—but are not limited to—the PCR amplification, sequencing such as 16S RNA or shotgun metagenomics present in a microbiota sample.

As used herein, a “microbiota sample” refers to any biological sample that can be used to detect the presence and the composition of the gut microbiota.

In an aspect, the present invention provides a method for determining whether an individual has or is at risk of having an alteration of the gut bacterial diversity, by determining the relative abundance of Faecalibacterium and Bacteroides in a microbiota sample.

In embodiments, said method comprises:
(i) determining the ratio of Faecalibacterium population/Bacteroides population in a microbiota sample from said individual,
(ii) determining that the individual has or is at risk of having an altered gut bacterial diversity, if the ratio of Faecalibacterium population/Bacteroides population is inferior or equal to 1/4.

In an embodiment, Faecalibacterium and Bacteroides populations are the two most abundant populations in the gut microbiome of the individual. Indeed, as shown by the Inventors, most of the alterations of gut bacterial diversity occur in the Bacteroides-enriched populations where Faecalibacterium and Bacteroides genera had the highest weight.

In a preferred embodiment, Bacteroides is the most abundant population in the gut microbiome of the individual as determined by relative abundance.

According to the invention, “the most abundant population” means that a genus is dominant among all genera usually detected.

In an embodiment, Bacteroides is the most abundant genus compared to the other genera usually detected, e.g. Faecalibacterium, Roseburia, Prevotella, Akkermansia, Bifidobacterium, Blautia, Coprococcus, Ruminococcus, Alistipes, Parabacteroides, Clostridium and Lachnospira.

The abundance of each bacterial genus can be determined by various techniques which are commonly used by a skilled person in the art, such as DNA or RNA sequencing.

Accordingly, an embodiment of the invention relates to the method as defined above which comprises a prior step of determining the composition of the gut microbiome.

In particular, an embodiment of the invention relates to the method as defined above which comprises a prior step of a) quantifying Bacteroides, and at least one or more selected from the group consisting of Faecalibacterium, Roseburia, Prevotella, Akkermansia, Bifidobacterium, Blautia, Coprococcus, Ruminococcus, Alistipes, Parabacteroides, Clostridium and Lachnospira; b) determining the relative abundance of each genus. In a preferred embodiment, the method as defined above is carried out only when Bacteroides is the most abundant genus.

In an embodiment, the invention relates to the method as defined above which further comprises:
(iii) providing a dietary recommendation to an individual at risk of having an altered gut bacterial diversity, suitable for increasing the ratio of Faecalibacterium population/Bacteroides population or administering to said individual a diet or a treatment in order to promote restoration of the gut bacterial diversity.

In an embodiment, the invention relates to the method as defined above which further comprises:
(iii) administering to said individual:
a diet enriched with fibers,
a diet comprising one or more probiotic species,
a food supplement comprising Faecalibacterium bacteria,
a diet enriched with Faecalibacterium bacteria,
a diet enriched with riboflavin (vitamin B2), and/or
a treatment for reducing oxidation and/or inflammation.

In an embodiment, the invention relates to the method as defined above which further comprises:
(iii) administering to said individual a diet enriched with fibers.

In an embodiment, the invention relates to the method as defined above which further comprises:
(iii) administering to said individual a diet comprising one or more probiotic species.

In an embodiment, the invention relates to the method as defined above which further comprises:
(iii) administering to said individual a food supplement comprising Faecalibacterium bacteria.

In an embodiment, the invention relates to the method as defined above which further comprises:
(iii) administering to said individual a diet enriched with Faecalibacterium bacteria.

In an embodiment, the invention relates to the method as defined above which further comprises:
(iii) administering to said individual a diet enriched with riboflavin (vitamin B2).

In an embodiment, the invention relates to the method as defined above which further comprises:
(iii) administering to said individual a treatment for reducing oxidation and/or inflammation.

In an embodiment, the invention relates to the method as defined above, wherein the diet enriched with fibers is a diet enriched with inulin, pectin and/or fructo-oligosaccharides (FOS).

In an embodiment, the invention relates to the method as defined above, wherein the diet administered to the individual comprises fermented dairy products or plant-based milk alternatives.

In an embodiment, the invention relates to the method as defined above, wherein said probiotics are chosen from the group comprising Streptococcus species, Lactococcus species and Bifidobacterium species.

In an embodiment, the invention relates to the method as defined above, wherein said probiotics are chosen from the group comprising Streptococcus thermophilus, Lactococcus lactis subsp. lactis, Bifidobacterium lactis, Bifidobacterium adolescentis, Bifidobacterium longum, Bifidobacterium breve, Bifidobacterium Bifidum, Bifidobacterium pseudocatenulatum, Lacticaseibacillus rhamnosus and Lacticaseibacillus paracasei.

In an embodiment, the invention relates to the method as defined above, wherein said probiotics are Streptococcus thermophilus.

In an embodiment, the invention relates to the method as defined above, wherein said probiotics are Lactococcus lactis subsp. lactis.

In an embodiment, the invention relates to the method as defined above, wherein said probiotics are Bifidobacterium lactis.

In an embodiment, the invention relates to the method as defined above, wherein said probiotics are Bifidobacterium adolescentis.

In an embodiment, the invention relates to the method as defined above, wherein said probiotics are chosen from the group comprising Streptococcus thermophilus CNCM I-3862, Lactococcus lactis subsp. lactis CNCM I-1631 and Bifidobacterium lactis CNCM I-2494.

CNCM I-1631 refers to the strain deposited at the Collection Nationale de Cultures de Microorganismes (CNCM) (Institut Pasteur, 25-28 Rue du Docteur Roux, 75724 Paris Cedex 15, France) under the Budapest Treaty on Oct. 24, 1995 under reference number CNCM I-1631.

CNCM I-2494 refers to the strain deposited at the CNCM under the Budapest Treaty on Jun. 20, 2000 under reference number CNCM I-2494.

CNCM I-3862 refers to the strain deposited at the CNCM under the Budapest Treaty on Oct. 31, 2007 under reference number CNCM I-3862.

In an embodiment, the invention relates to the method as defined above, wherein the individual has or is at risk of having an altered gut bacterial diversity, if the ratio of Faecalibacterium population/Bacteroides population is inferior or equal to 1/5, 1/10, 1/20, 1/30, 1/40, 1/50, 1/60, in particular inferior or equal to 1/64.

In an embodiment, the invention relates to the method as defined above, wherein the individual has or is at risk of having an altered gut bacterial diversity, if the ratio of Faecalibacterium population/Bacteroides population is ≤1/4 and >1/64.

It is expected that a dietary intervention would be useful to restore the gut diversity when the Faecalibacterium/Bacteroides ratio is ≤1/4 and >1/64. For example, a dietary intervention can be—but is not limited to—the administration of a diet comprising one or more probiotic species and/or comprising enriched with Faecalibacterium bacteria and/or enriched with fibers and/or enriched with riboflavin (vitamin B2) to the individual.

In an embodiment, the invention relates to the method as defined above wherein the individual has or is at risk of having an altered gut bacterial diversity, if the ratio of Faecalibacterium population/Bacteroides population is ≤1/64.

It is expected that a therapeutic approach could be required to restore the gut diversity when the Faecalibacterium/Bacteroides ratio is ≤1/64. For example, a therapeutic approach can be—but is not limited to—the administration of a treatment for reducing oxidation and/or inflammation and/or some antibiotics targeting Bacteroides bacteria to the individual.

The microbiota sample can be a fecal sample or a mucosal sample, preferably a fecal sample.

In an embodiment, the invention relates to the method as defined above, which comprises a step of isolating the microbiota sample before step (i). The isolation of the microbiota sample can achieved by non-invasive procedures, e.g. collecting a fecal sample, or more invasive procedures, e.g. collecting mucosal samples via a colonoscopy or a biopsy.

In an embodiment, the invention relates to the method as defined above, which comprises a step of analyzing the microbiota sample before step (i). The analysis of the microbiota sample can be achieved by various techniques, such as a PCR amplification of genomic sequences present in the sample or by plating on selective media.

All the embodiments and features given above apply mutatis mutandis to the other following aspects of the invention.

In another aspect, the present invention also relates to the use of the ratio of Faecalibacterium population/Bacteroides population in a microbiota sample as a biomarker for determining whether an individual has or is at risk of having an alteration of the gut bacterial diversity.

In another aspect, the present invention thus provides a method for determining whether an individual has or is at risk of having a gut dysbiosis, by determining the relative abundance of Faecalibacterium and Bacteroides in a microbiota sample, said method comprising:
(i) determining the ratio of Faecalibacterium population/Bacteroides population in a microbiota sample from said individual,
(ii) determining that the individual has or is at risk of having a gut dysbiosis, if the ratio of Faecalibacterium population/Bacteroides population is inferior or equal to 1/4.

In another aspect, the present invention also relates to the use of the ratio of Faecalibacterium population/Bacteroides population in a microbiota sample as a biomarker for determining whether an individual has or is at risk of having a gut dysbiosis.

The invention will be further illustrated by the following non-limiting Figures and Examples.

### DESCRIPTION OF THE FIGURES

FIG. 1. Grouping the partitions according to their similarity in American Gut Project. Each partition centroids are mapped on the branches.

FIG. 2. Longitudinal analysis of DMM partitions within the branches. a. A network between partitions shows how individuals can change partition as a function of time. Node size indicates the stability of the partitions, and the intensity reflects the corresponding branches. Arrow width represents the proportion of change between each node. Only edges representing more than 5% of the partition events (including switch and non-switch), to decrease visual noise, were shown representing 81% of the events.

FIG. 3. Faecalibacterium:Bacteroides ratio distribution density within Bacteroides-enriched branch. The vertical black line shows the average Faecalibacterium:Bacteroides ratio for AGP participants that switched to another partition over time (h) Faecalibacterium:Bacteroides ratio as a function of Bacteroides-enriched partitions.

FIG. 4. Faecalibacterium:Bacteroides ratio from AGP gut microbiome sample classified in Bacteroides enriched branch as a function of gut microbiome alpha-diversity (shannon index). Microbiota DMM partitions classified thank to Faecalibacterium:Bacteroides ratio threshold (dotted lines) defined from longitudinal data analysis.

FIG. 5. Partition M1 individuals characteristics. Barplot showing the prevalence of a selection of AGP questionnaire answers in all AGP participants compared to AGP belonging to the gut microbiome partition M1. Partition M1, the highest alpha diversity and the structural root of branches, was used as the reference for multinomial logistic regression analysis.

FIG. 6. Absolute model weights by predictor used in the model depicted among Bacteroides dominant partitions. Partitions were separated based on Faecalibacterium:Bacteroides ratio clusters. Heatmap showing predictor log odd ratio (OR) as a function of partition. Significant OR were shown with a black dot (FDR<10%). Binary predictors, like disease self-declaration, were coded by 0 to 1 (e.g., “Diagnosed by a medical professional” encoded to 1, “I do not have this conditions” encoded to 0, other categories were considered as missing values). Color intensity >0.0 to 2.5 indicates that partition is associated with higher frequency of disease. For diet, like food groups frequency, were coded from 0 (never) to 1 (daily). Color intensity<0.0 to −2.5 indicates that partition is associated with lower frequency of intake. Antibiotic history was considered as an ordered factor from “I have not taken antibiotics in the past year” to antibiotic intake within the “week” of fecal sample collection. Color intensity >0.0 to 2.5 indicates that partition is associated with shorter time since last antibiotic intake.

### EXAMPLES

### Methods

### American Gut Project (AGP) Dataset

In the AGP initiative, stool samples were collected at home and shipped at room temperature before microbial DNA extraction and 16S rRNA amplicon sequencing, which were performed as previously described (McDonald et al., mSystems, 3:e00031-18, 2018). The Inventors used redbiom (McDonald et al., mSystems, 4:e00215-e00219, 2019) to fetch data from Qiita (Gonzalez et al., Nature methods, 15:796-98, 2018). 20,454 stool sample identifiers were available in the database on the date 2019 Dec. 5, within the Deblur-Illumina-16S-V4-100nt-fbc5b2 context. Analyses were performed as previously described (Cotillard et al., 2021). In short, bioinformatic analysis was performed with QIIME 2019.10, bloom sequences were removed as previously described (Amir et al., mSystems, 2:e00199-16, 2017), and taxonomy was assigned using the GreenGenes database (v 13.5). The Inventors retained samples>=1,000 reads. 1,579 samples were defined as technical outliers and excluded following the criteria in Cotillard et al., 2021. A genus count matrix with 16 021 samples was analyzed.

### Gut Microbiome Partitioning and Branches

In AGP, the 30 genera with the highest read mass were extracted for downstream analyses. Samples were partitioned using Dirichlet's multinomial mixture (DMM) modeling on the microbiota data (Holmes et al., PloS one 7: e30126, 2012). The Inventors trained DMM models using five subsets from the whole dataset to reduce population bias. Each subset was sampled from the whole dataset with stratification by each combination of sex, geographic region of origin (region of birth for the AGP dataset), and age class (n up to 30 by strata). Subsampling was not limited to a single sample per subject. Of note, participants having at least two samples represented less than 5% of the participants. DMM modeling was performed for each subset constituting our training set, and the best model was picked up using BIC and Laplace minima, and majority vote (i.e., which optimal value of the k parameter was picked up more often). Partitions homogeneity were assessed using theta index extracted from DMM models. Low values of theta correspond to highly variable partitions. The whole dataset was modeled with DMM using the corresponding genera from the training dataset in the remaining dataset. Genera alpha weights for each DMM component from the AGP datasets were compared using hierarchical clustering on the Jensen-Shannon distance (Ward's method).

The Inventors assessed microbiome branch latent structure on and AGP dataset (genus relative abundance) using the PHATE algorithm (phateR version 1.0.7), with the gamma parameter set to zero (Moon et al., Nature Biotechnology 37: 1482-92, 2019) for visualization purposes and all other parameters set to default. Based on DMM genera alpha weight, the Inventors extracted microbiome branches.

### Longitudinal Analysis

To assess stability patterns within partitions and switching between partitions over time, The Inventors extracted data from 745 participants of the AGP dataset who had provided at least two samples, resulting in 2,998 samples. Partition stability was assessed by comparing the proportion of individuals who remained in their initial assigned partition and the proportion of individuals who remained in their randomly assigned partition. 100 random assignations were performed to compute a confidence interval for each partition. The percentile 95^{th} for stability obtained from randomized assigned partitions was retained as a threshold for significatively.

### Health, Demographic, and Dietary Associations With Partitions and Branches

To assess whether health, demographic, and diet participants' metadata were associated with gut microbiome partitions and branches in AGP, the Inventors fitted a multinomial log-linear model via neural networks using the nnet R package (version 7.3-15). Gut microbiome partitions were used as responses and metadata as predictors. The partition with the highest alpha-diversity assessed with the Shannon index was defined as the reference.

Categorical variables, like region of birth, were one-hot encoded. Binary predictors, like disease self-declaration, were coded by 0 to 1 (e.g., “Diagnosed by a medical professional” encoded to 1, “I do not have this conditions” encoded to 0, other categories were considered as missing values). Continuous predictors, like Age and BMI, were scaled from 0 to 1. Ordered predictors, like food groups frequency, were coded from 0 (never) to 1 (daily). Antibiotic history was considered as an ordered factor from “I have not taken antibiotics in the past year” to antibiotic intake within the “week” of fecal sample collection. In short, 100 health, demographic and dietary predictors were used to build the model. Missing values were replaced by the average computed for each predictor.

Log odds ratios by predictor and gut microbiome partition and their respective p-values were extracted from the resulting model using broom helpers R package (version 1.2.1). False discovery rate was computed by predictors. All statistical analyses were computed using R software version 3.6 (Team 2021).

### Results

To identify partitions of the human gut microbiome, i.e., possible ecological states, the Dirichlet Multinomial Mixtures (DMM) partitioning method was used since it was extensively used in prior attempts in gut microbiome studies. The Potential of Heat-diffusion for Affinity-based Trajectory Embedding (PHATE) algorithm was applied to further explore this possibility. PHATE is a visualization method conceived to discover latent structures, such as transitions, in high dimensional data while conserving global and local structures of the data, which has previously been applied to gut microbiome data to detect “branches” (Moon et al., 2019). The partitioning and ordination approach was applied to the American Gut Project dataset, which consists of ca. 16,000 16S rRNA gene amplicon sequencing-based fecal samples associated with multiple demographic, lifestyle, health, dietary variables, and which includes longitudinal sampling from a subset of individuals. Using DMM-based modeling at the genus level, the best model fit in the AGP database was obtained with 19 partitions. High intra-partitions homogeneity (except for partition 19) based on high prediction confidence and consistent Shannon alpha diversity between the training and remaining sets were shown.

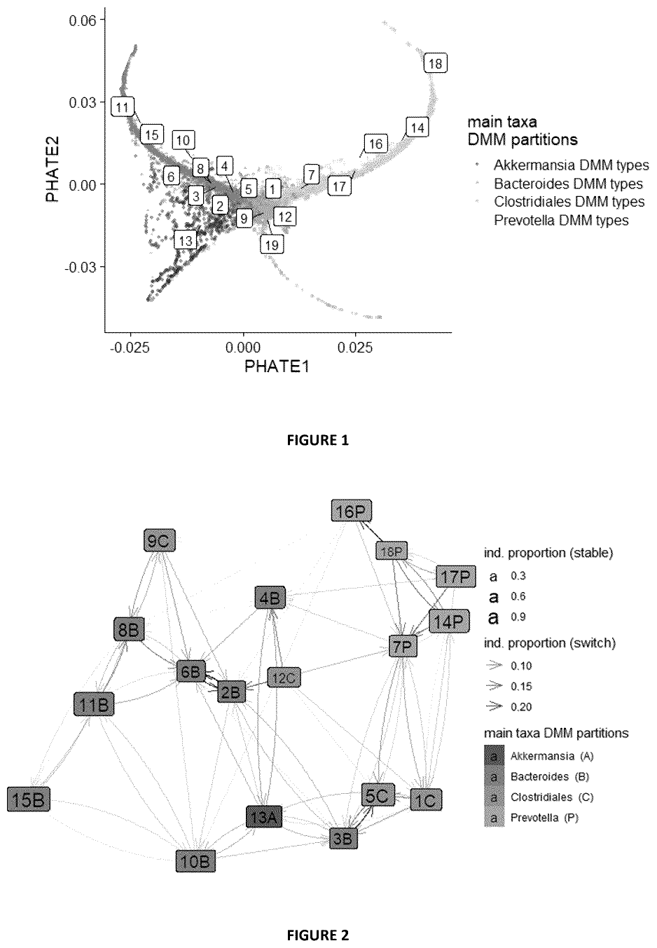

Then, a PHATE-map of the AGP dataset was generated and resulted in the generation of three main branches. A contrasting Bacteroides to Prevotella ratio discriminated the major two branches. The projection of the centroids of the 19 DMM-partitions on this PHATE-map led to partitions being arranged along the global branches. It is noteworthy to observe a declining gradient of alpha-diversity from Clostridiales-dominated partitions towards the tips of the Bacteroides or Prevotella branches: the M1 partition being the most diverse while the least diverse Bacteroides and Prevotella partitions, respectively M11 and M18, formed the tips of their branches (FIG. 1).

Then, data originating from 745 participants of the AGP cohort, from whom at least two samples were collected over time with 12.5 (IQR [1.1-73.0]) days apart on average, were analysed. Each of the associated 2,998 gut microbiome profiles were associated to a partition and a branch and the changes between two-time points were reported. Then, to test whether local partitions are either random stratification or ecological states, a network was built based on longitudinal data as a function of stability, with instability marked by a high occurrence of observed switches (FIG. 2). Of note, this network was performed in unsupervised way, (i.e not integrating the order of partitions depicted in FIG. 1).

The percentage of individuals who remained in their initial partition was, on average 42%, and consistently superior to the frequency computed using randomly generated events (~10% upper CI 95% bound), suggesting that partitions may be relatively stable states. Out of the 18 partitions, the M15 and M14 partitions were the most stable within the Bacteroides and Prevotella branches, respectively. In contrast, some partitions were connected by a higher occurrence of switches (e.g., between M3 and M5 and between M2 and M6) based on the network. These switches occurred in the Bacteroides-enriched branch where Faecalibacterium and Bacteroides genera had the highest weight in those partitions. These partitions were further assessed to determine whether they resulted from an artifact of over-partitioning. It was reasoned that an over-partitioning would result in the same variation over time between and within tested partitions using alpha-diversity and the ratio of the two most abundant genera (Faecalibacterium and Bacteroides) as markers of the ecosystem composition in the Bacteroides-enriched branch. By plotting the variation over time of those parameters, it was observed that compositional changes were limited where switching between the partitions did not occur by an individual. Meanwhile, compositional changes were scattered and overlapping where switching did occur between partitions.

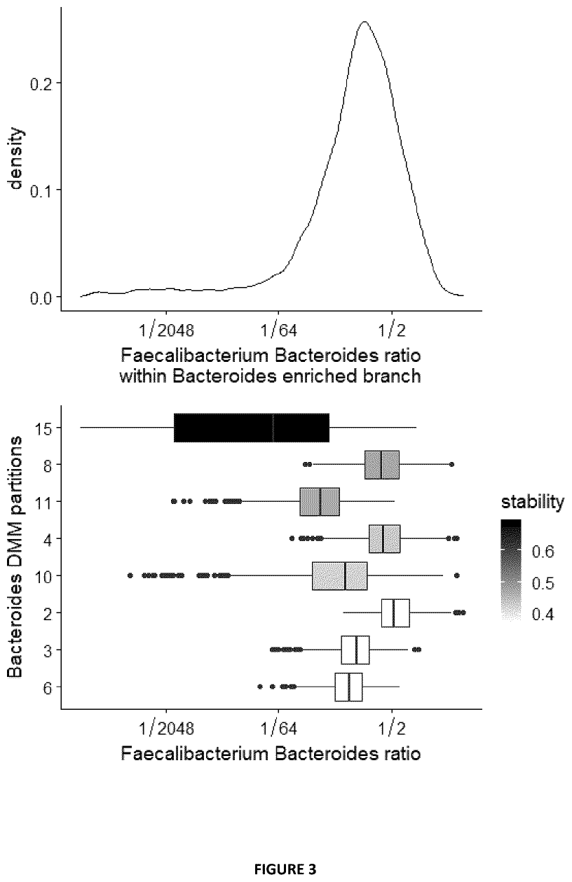

The dynamics of the Faecalibacterium:Bacteroides ratio during M3/M5 and M2/M6 partition switches were further investigated and it was observed that the variations of this ratio were elevated in the case of inter-partition switches compared to intra-partition fluctuations. This observation suggests that the differential ratio of Faecalibacterium:Bacteroides is larger between samples of participants that switch between partitions than those that remain stable over time within a partition, supporting our hypothesis that partitions may represent local stable states. Of note, the median values of the Faecalibacterium:Bacteroides ratio in subjects that switch partitions over time nearly coincided between the two sets of partitions (M2/M6 and M3/M5), at 0.24 and 0.26 respectively, possibly suggesting that a Faecalibacterium:Bacteroides ratio of 1:4 may be a potential marker of gut microbiome instability (FIG. 3) for the Bacteroides-enriched microbiome branch. Based on this 1:4 ratio, M8, M4, and M2 were classified as Faecalibacterium:Bacteroides high ratio group. Partition M15 have more than 50% of sample having a Faecalibacterium:Bacteroides ratio lower than 1:64. Partition M15 can be then classified as Faecalibacterium:Bacteroides low ratio group. Other partitions (M6, M3, M10, and M11) that belonged to Bacteroides enriched branch were classified in the Faecalibacterium:Bacteroides low ratio group.

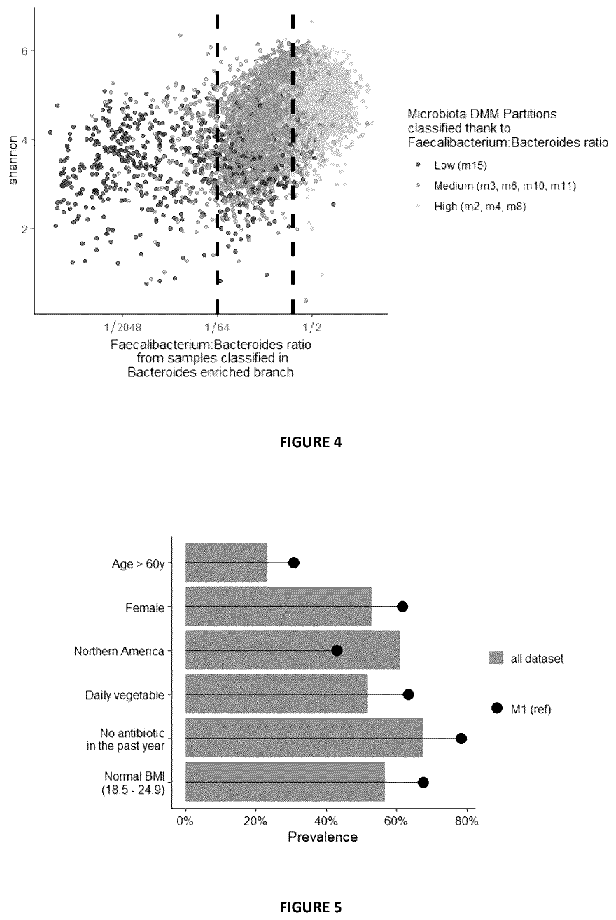

For the partitions with medium and low Faecalibacterium:Bacteroides ratio, there was a significant correlation with alpha-diversity. Notably, they had higher Shannon index (FIG. 4).

The AGP dataset (ca 16.000 fecal samples) was interrogated to identify associations between branches/partitions and factors relating to the host including dietary habits, lifestyle, region of birth, age, BMI, bowel movement frequency, sex, diseases and antibiotic history). Given the high dimensional nature of the data, a multinomial logistic regression across 100 predictors (i.e., factors) collected through the AGP main questionnaire (FDR<0.1) was fitted. To gain insights on factors that could explain the decreased diversity of the communities along the branches, the most central and diverse partition (M1) was used as the reference in this logistic regression, which was primarily composed of female participants consuming vegetables in high frequency (daily), with low exposure to antibiotics (FIG. 5). For each predictor, the model returned odds ratios representing the strength of association for a given partition (vs. the reference one).

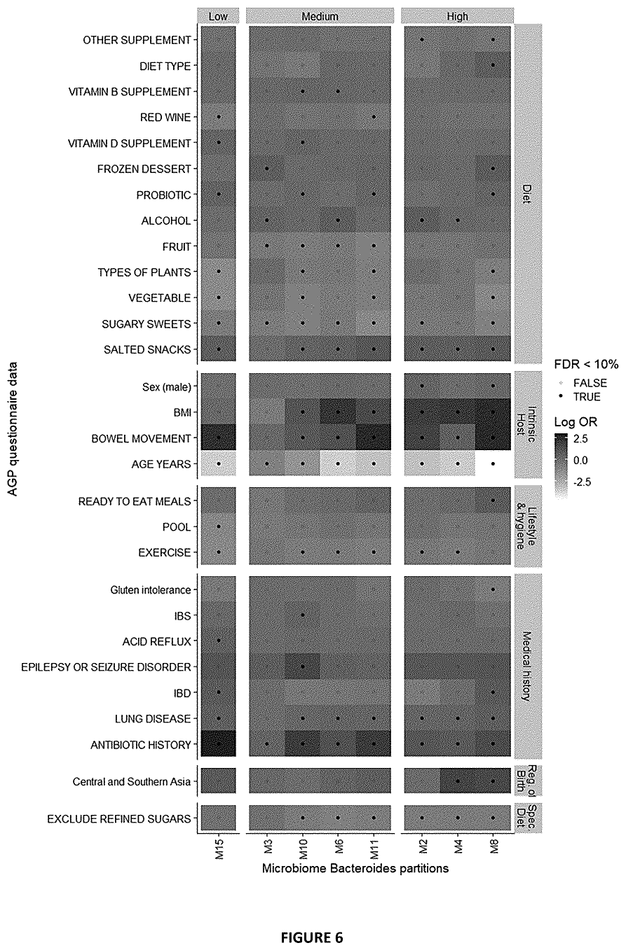

Individuals from the Faecalibacterium:Bacteroides low and medium ratio group were mainly associated with lower vegetable, fruit, and plant diversity consumption in their diet. The Faecalibacterium:Bacteroides low ratio group were mainly associated with higher antibiotic intakes and diseases (FIG. 6).

Taken together, our data showed that partitions exhibited both common and differential characteristics for both environmental and host factors that were partition-dependent.

A ratio lower or equal than 1/4 and >1/64 might be targeted by dietary intervention, while <1/64 by therapeutic approaches.

1. A method for determining whether an individual has or is at risk of having an alteration of the gut bacterial diversity, comprising determining the relative abundance of Faecalibacterium and Bacteroides in a microbiota sample and providing a dietary recommendation to an at risk individual suitable for increasing the ratio of Faecalibacterium population/Bacteroides population.

2. A method for determining whether an individual has or is at risk of having an alteration of the gut bacterial diversity, said method comprising:
(i) determining the ratio of Faecalibacterium population/Bacteroides population in the microbiota sample from said individual, and
(ii) determining that the individual has or is at risk of having an altered gut bacterial diversity, if the ratio of Faecalibacterium population/Bacteroides population is inferior or equal to 1/4.

3. A method for determining whether an individual suffers from or is at risk of developing a gut dysbiosis, said method comprising:
(i) determining the ratio of Faecalibacterium population/Bacteroides population in the microbiota sample from said individual, and
(ii) determining that the individual suffers from or is at risk of developing a gut dysbiosis, if the ratio of Faecalibacterium population/Bacteroides population is inferior or equal to 1/4.

4. The method according to claim 1, wherein Faecalibacterium and Bacteroides populations are the two most abundant populations in the gut microbiota of the individual.

5. The method according to claim 1, wherein the microbiota sample is a fecal sample.

6. The method according to claim 1, further comprising:
administering to said individual a diet or a treatment in order to promote restoration of the gut bacterial diversity.

7. The method according to claim 1, comprising:
administering to said individual:
a diet enriched with fibers,
a diet comprising one or more probiotic species,
a food supplement comprising Faecalibacterium bacteria,
a diet enriched with Faecalibacterium bacteria,
a diet enriched with riboflavin (vitamin B2), and/or
a treatment for reducing oxidation and/or inflammation.
